(19) United States
(12) Patent Application Publication
(10) Pub. No.: US 2016/0237495 A1
(43) Pub. Date: Aug. 18, 2016

(54) METHODS FOR DIAGNOSING CHRONIC VALVULAR DISEASE

(71) Applicant: NESTEC SA, Vevey (CH)

(72) Inventor: Qinghong Li, Chesterfield, MO (US)

(21) Appl. No.: 15/015,318

(22) Filed: Feb. 4, 2016

Related U.S. Application Data
(60) Provisional application No. 62/117,189, filed on Feb. 17, 2015.

(51) Int. Cl. C12Q 1/68

(52) U.S. Cl. CPC C12Q 1/6883; C12Q 2600/178

(57) ABSTRACT

The invention provides methods for diagnosing chronic valvular disease in an animal. The methods comprise obtaining a sample from the animal; analyzing the sample for an amount of a microRNA (miRNA) associated with chronic valvular disease; comparing the amount of the miRNA identified in the sample to a corresponding amount of the miRNA present in a sample from one or more comparable control animals that do not suffer from chronic valvular disease; and diagnosing the animal with chronic valvular disease if the amount of the miRNA found in the animal's sample is differentially expressed in the control animal's sample.

### CROSS REFERENCE TO RELATED APPLICATIONS

This application claims priority to U.S. Provisional Application No. 62/117189 filed Feb. 17, 2015, the disclosure of which is incorporated herein by this reference.

### BACKGROUND OF THE INVENTION

1. Field of the Invention

The invention relates generally to methods for diagnosing and predicting chronic valvular disease and particularly to methods for diagnosing and predicting chronic valvular disease by measuring microRNA (miRNA) associated with chronic valvular disease.

2. Description of Related Art

Cardiac disease is one of the most common disorders in dogs. Chronic Valvular Disease (CVD), also known as degenerative mitral disease (DMVD), affects approximately 9% of all dogs, increasing with age such that the overall cumulative incidence is greater than 40%. CVD is characterized by a progressive degeneration and deformation of the atrioventricular valves, most commonly the mitral valves, resulting in early mitral valve insufficiency. This in turn leads to the appearance of a systolic heart murmur due to mitral regurgitation, wherein inadequate closure of the mitral valve causes blood to flow back to the left atrium. The affected dogs finally develop left atrioventricular volume overload, pulmonary edema, atrial dilatation and supraventricular arrhythmias.

Although surgical or medical treatment of affected valves is possible, nutritional intervention is preferred by pet owners. Early detection and treatment are imperative however detection can be difficult due to the lack of symptoms. A patent application (PCT/US201344011) describes methods for diagnosing CVD using differentially expressed genes and metabolites. However, gene expression methods require collection of cardiac tissues which is considered invasive. In addition, mRNA is not very stable and can be difficult to handle experimentally. Further, metabolite measurement from blood serum can be difficult depending on the biochemical nature and stability of the compounds.

Therefore there remains a need for diagnosing and predicting CVD in animals to provide the most appropriate and effective level of treatment.

### SUMMARY OF THE INVENTION

It is, therefore, an object of the present invention to provide methods for diagnosing and predicting chronic valvular disease in animals.

This and other objects are achieved using methods for diagnosing and predicting chronic valvular disease in an animal that involve obtaining a sample from the animal; analyzing the sample for an amount of an miRNA associated with chronic valvular disease; comparing the amount of the miRNA identified in the sample to a corresponding amount of the miRNA present in a sample from one or more comparable control animals that do not suffer from chronic valvular disease; and diagnosing the animal with chronic valvular disease if the miRNA found in the animal's sample is differentially expressed in the control animal's sample.

Other and further objects, features, and advantages of the present invention will be readily apparent to those skilled in the art.

### DETAILED DESCRIPTION OF THE INVENTION

### Definitions

The term “animal” means any animal susceptible to or suffering from chronic valvular disease, including human, avian, bovine, canine, equine, feline, hircine, lupine, murine, ovine, or porcine animals.

The terms “miRNA” or “biomarker” mean a small single-stranded non-coding RNA molecule, including those containing about 21-25 nucleotides, the levels or intensities of which are measured in a biological sample, that may be used as markers to diagnose a disease state.

The term “differential expression” or “differentially expressed” means increased or upregulated miRNA expression or means decreased or downregulated miRNA expression as detected by the absence, presence, or change in the amount of miRNA in a sample.

The term “comparable control animal” means an animal of the same species and type or an individual animal evaluated at two different times.

The term “corresponding amount” means an amount of an miRNA from a comparable control animal that corresponds to the miRNA for an animal being diagnosed with chronic valvular disease, where the miRNA is associated with chronic valvular disease.

The term “companion animals” means domesticated animals such as dogs, cats, birds, rabbits, guinea pigs, ferrets, hamsters, mice, gerbils, pleasure horses, cows, goats, sheep, donkeys, pigs, and more exotic species kept by humans for company, amusement, psychological support, extrovert display, and all of the other functions that humans need to share with animals of other species. In one aspect, companion animal can refer to a dog or cat. In one specific aspect, a companion animal can refer to a dog.

The term “diagnosing” means determining if an animal is suffering from or predicting if the animal is susceptible to developing chronic valvular disease.

The term “early stage” means stage B heart failure (HF) with mild to moderate cardiac enlargement but no clinical signs of heart failure (HF) based upon the guidelines for diagnosis of HF from American College of Veterinary Internal Medicine (ACVIM).

The term “end stage” means stage C HF with clinical signs of moderate HF based upon the ACVIM guidelines.

As used herein, ranges are used herein in shorthand, so as to avoid having to list and describe each and every value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a method” includes a plurality of such “methods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

### The Invention

The present inventors have discovered that miRNA described herein can be present in the biological sample of an animal and that the amount of the miRNA in the sample can serve as a biochemical indicator for diagnosing chronic valvular disease by indicating or predicting the threshold for chronic valvular disease. As such, the present discovery allows veterinary and other clinicians to perform tests for these “biomarkers” in a sample and determine whether the animal is susceptible to or suffering from chronic valvular disease and whether there is a need for further diagnostics or treatment. Having established the need for further diagnostics or treatments, the cost and risk of such further diagnostics or treatments can be justified.

In accordance with the above, the invention provides methods for diagnosing chronic valvular disease in an animal. The methods comprise obtaining a biological sample from the animal, analyzing the sample for an amount of a microRNA (miRNA) associated with chronic valvular disease, comparing the amount of the miRNA identified in the sample to a corresponding amount of the miRNA present in a sample from one or more comparable control animals that do not suffer from chronic valvular disease, and diagnosing the animal with chronic valvular disease if the amount of the miRNA found in the animal's sample is differentially expressed in the control animal's sample.

In one embodiment, a method for diagnosing chronic valvular disease in an animal can comprise obtaining a biological sample from the animal, analyzing the sample for an amount of an miRNA associated with chronic valvular disease, comparing the amount of the miRNA identified in the sample to a corresponding amount of the miRNA present in a sample from one or more comparable control animals that do not suffer from chronic valvular disease, and diagnosing the animal with chronic valvular disease if the amount of the miRNA found in the animal's sample is greater than the amount of the miRNA present in the control animal's sample.

In another embodiment, a method for diagnosing chronic valvular disease in an animal can comprise obtaining a biological sample from the animal, analyzing the sample for an amount of an miRNA associated with chronic valvular disease, comparing the amount of the miRNA identified in the sample to a corresponding amount of the miRNA present in a sample from one or more comparable control animals that do not suffer from chronic valvular disease, and diagnosing the animal with chronic valvular disease if the amount of the miRNA found in the animal's sample is less than the amount of the miRNA present in the control animal's sample.

In various embodiments, one or more comparable control animals that are not the animal being evaluated for chronic valvular disease and that have been determined not to suffer from chronic valvular disease can be evaluated for the miRNA and the results of such evaluations are used as a baseline value for comparison with the results from an animal being evaluated for the miRNA. In some embodiments, the baseline value for the miRNA can be determined by evaluating numerous comparable control animals.

In another embodiment, the amount of miRNA can be determined for an animal at various times throughout the animal's life and the results can be used to determine if the animal is susceptible to or suffering from chronic valvular disease, e.g., if the amount of the miRNA increases or decreases as the animal ages, the animal can be diagnosed as susceptible to or suffering from chronic valvular disease. In some embodiments, the animal can be evaluated periodically and the results for the miRNA can be recorded. Then, if a subsequent evaluation shows that the amount of the miRNA has increased or decreased since the last evaluation(s), the animal can be diagnosed as susceptible to or suffering from chronic valvular disease. In some aspects, specific changes in the miRNAs can be correlated to an early stage or end stage of chronic valvular disease.

Any sample that is of biological origin may be useful in the present invention. Examples include, but are not limited to, blood (serum/plasma), cerebral spinal fluid (CSF), urine, stool, breath, saliva, or biopsy of any tissue. In one embodiment, the sample can be a serum sample. While the term “serum” is used herein, those skilled in the art will recognize that plasma or whole blood or a sub-fraction of whole blood may also be used.

Decreased or increased expression can be measured at the miRNA level using any of the methods well known in the art for the quantitation of polynucleotides, such as, for example, PCR (including, without limitation, RT-PCR and qPCR), sequencing, Northern blotting, microarray, or other hybridization methods.

While the use of one miRNA is sufficient for diagnosing chronic valvular disease, the use of one or more, two or more, three or more, or four or more of such miRNA is encompassed within the invention. The miRNA can be evaluated and used for a diagnosis in any combination. As such, the present methods can include diagnosing CVD based an upregulated miRNA and a downregulated miRNA.

In one embodiment, the diagnosis can be based upon determining if the amount of the miRNA found in the animal's sample is greater compared to the amount of the miRNA present in the control animal's sample. In one aspect, the miRNA can include miR-103, miR-98, let-7c, or let-7b.

In one embodiment, the diagnosis can be based upon determining if the amount of the miRNA found in the animal's sample is less than compared to the amount of the miRNA present in the control animal's sample. In one aspect, the miRNA can include miR-302d, miR-380, miR-874, miR-582, miR-490, miR-329b, or miR-487b.

The differentially expressed miRNA can be statistically significant as exemplified herein. In some aspects, the statistical significance can include a p<0.05, p<0.01, or even p<0.001.

In various embodiments, the animal can be a human or companion animal. In one aspect, the companion animal can be a canine such as a dog or a feline such as a cat. In one specific aspect, the animal can be a canine.

### EXAMPLES

The invention can be further illustrated by the following examples, although it will be understood that these examples are included merely for purposes of illustration and are not intended to limit the scope of the invention unless otherwise specifically indicated.

### Example 1

miRNAs were identified through a differential expression profiling study comparing diseased and normal serum samples. Dogs were classified as having either a healthy heart or CVD by echocardiography performed or evaluated by a board-certified veterinary cardiologist, pathological examination of the heart or both. Dogs were classified into one of the three groups based upon their stage of heart failure (HF) using the American College of Veterinary Internal Medicine (ACVIM)/European College of Veterinary Internal Medicine (ECVIM) staging scheme: group A (stage A, at risk but unaffected, 6 dogs), group B (stage B, with mild to moderate cardiac enlargement but no clinical sign of heart failure, 6 dogs), or group C (stage C, with clinical sign of moderate heart failure, 6 dogs) (Table 1). These three groups of dogs were matched by age, body size, and sex. Blood serum was collected from each group of dogs, and was stored in −80° C. until use.

TABLE 1
Serum samples used in miRNA study

| Group | Number | Disease stage classification |
|---|---|---|
| A | 6 | ACVIM stage A. At risk but |
| | | unaffected |
| B | 6 | ACVIM stage B. Mild to |
| | | moderate cardiac |
| | | enlargement, but no clinical |
| | | sign of HF |
| C | 6 | ACVIM stage C. Clinical |
| | | sign of moderate HF. |

Samples were submitted to Qiagen for miRNA extraction and PCR quantification using Qiagen's miScript® miRNA PCR array system. A total of 277 known canine miRNAs were assayed. Data analysis was performed using ANOVA (Analysis of Variance). The ANOVA P values were adjusted for multiple testing and the false discovery rate (FDR) was calculated using the Benjamini and Hochberg method. Expression fold change between groups B vs. A (FC_BvA), and between groups C vs. A (FC_CvA) were calculated. Differentially expressed miRNAs were selected using the threshold of FDR<0.05. A total of 11 miRNAs were selected (Table 2).

TABLE 2
Differentially expressed miRNAs

| miRNA | P^{a} | FDR^{b} | FC^{c}_BvA | FC^{c}_CvA |
|---|---|---|---|---|
| miR-302d | 0.0010 | 0.0378 | −2.7 | −3.51 |
| miR-380 | 0.0001 | 0.0119 | −2.48 | −4.76 |
| miR-874 | 0.0016 | 0.0434 | −2.37 | −3.69 |
| miR-582 | 0.0000 | 0.0004 | −2.28 | −4.71 |
| miR-490 | 0.0005 | 0.0261 | −2.24 | −4.03 |
| miR-329b | 0.0019 | 0.0490 | −1.96 | −2.36 |
| miR-487b | 0.0012 | 0.0427 | −1.45 | −3.08 |
| miR-103 | 0.0002 | 0.0140 | 1.29 | 3.81 |
| miR-98 | 0.0014 | 0.0428 | 2.15 | 4.07 |
| let-7c | 0.0003 | 0.0218 | 2.28 | 4.67 |
| let-7b | 0.0006 | 0.0269 | 2.38 | 4.03 |

^{a}ANOVA nominal P values;
^{b}false discovery rate;
^{c}fold changes positive numbers indicate upregulation from the control samples; negative numbers indicate downregulation from the control samples.

In the specification, there have been disclosed typical preferred embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims, the invention may be practiced otherwise than as specifically described.

What is claimed is:

1. A method for diagnosing chronic valvular disease in an animal comprising:
a. obtaining a biological sample from the animal;
b. analyzing the biological sample for an amount of a microRNA (miRNA) associated with chronic valvular disease;
c. comparing the amount of the miRNA identified in the sample to a corresponding amount of the miRNA present in a sample from one or more comparable control animals that do not suffer from chronic valvular disease; and
d. diagnosing the animal with chronic valvular disease if the amount of the miRNA found in the animal's sample is greater than the amount of the miRNA present in the control animal's sample.

2. The method of claim 1, wherein the sample is a serum sample.

3. The method of claim 1, wherein the diagnosis is based upon determining the amount of two or more miRNAs associated with chronic valvular disease.

4. The method of claim 1, wherein the diagnosis is based upon determining the amount of three or more miRNAs associated with chronic valvular disease.

5. The method of claim 1, wherein the diagnosis is based upon an amount of a second miRNA that is downregulated.

6. The method of claim 1, wherein the miRNA is selected from the group consisting of miR-103, miR-98, let-7c, let-7b, and combinations thereof.

7. The method of claim 1, wherein the animal is a companion animal.

8. The method of claim 7, wherein the companion animal is a canine.

9. A method for diagnosing chronic valvular disease in an animal comprising:
a. obtaining a biological sample from the animal;
b. analyzing the biological sample for an amount of a microRNA (miRNA) associated with chronic valvular disease;
c. comparing the amount of the miRNA identified in the sample to a corresponding amount of the miRNA present in a sample from one or more comparable control animals that do not suffer from chronic valvular disease; and
d. diagnosing the animal with chronic valvular disease if the amount of the miRNA found in the animal's sample is less than the amount of the miRNA present in the control animal's sample.

10. The method of claim 9, wherein the sample is a serum sample.

11. The method of claim 9, wherein the diagnosis is based upon determining the amount of two or more miRNAs associated with chronic valvular disease.

12. The method of claim 9, wherein the diagnosis is based upon determining the amount of three or more miRNAs associated with chronic valvular disease.

13. The method of claim 9, wherein the diagnosis is based upon an amount of a second miRNA that is upregulated.

14. The method of claim 9, wherein the miRNA is selected from the group consisting of miR-302d, miR-380, miR-874, miR-582, miR-490, miR-329b, miR-487b, and combinations thereof.

15. The method of claim 9, wherein the animal is a companion animal.

16. The method of claim 15, wherein the companion animal is a canine.
